(19) United States
(12) Patent Application Publication
(10) Pub. No.: US 2026/0268161 A1
(43) Pub. Date: Sep. 10, 2026

(54) EDGE PROCESSING OF PHYSIOLOGICAL DATA

(71) Applicant: Whoop, Inc., Boston, MA (US)

(72) Inventor: Mostafa Ghannad-Rezaie, Waltham, MA (US)

(21) Appl. No.: 19/562,449

(22) Filed: Mar. 10, 2026

Related U.S. Application Data
(60) Provisional application No. 63/769,536, filed on Mar. 10, 2025.

(51) Int. Cl. G06N 3/096; G06F 1/16; G16H 40/67

(52) U.S. Cl. CPC G06N 3/096; G06F 1/163; G16H 40/67

(57) ABSTRACT

A large foundation model is trained on a representative data set such as accumulated raw data from the sensors of a physiological monitor for a population of users. A distilled model is generated from the large foundation model and trained to identify information in a data stream from a physiological monitor that would be redundant or uninformative to the large foundation model. The distilled model can then execute on an edge device where it samples a data stream and filters out redundant or low-quality data from a source so that only portions of the data stream useful for inferencing are transmitted to the remote foundation model for processing.

### RELATED APPLICATIONS

This application claims priority to U.S. App. No. 63/769,536 filed on Mar. 10, 2025, the entire content of which is hereby incorporated by reference.

This application is also related to International Patent App. No. PCT/US26/18554 filed on Mar. 10, 2026, which claims priority to U.S. App. No. 63/769,536 filed on Mar. 10, 2025, where the entire content of each of the foregoing is hereby incorporated by reference.

### BACKGROUND

Large machine learning models are useful for analyzing data; however, when trained on very large data sets, these models often have correspondingly large storage and compute requirements that can render them unsuitable for edge computing. On the other hand, wearable devices that might use such models produce substantial amounts of data when continuously monitoring a user, making it difficult to transmit raw data feeds to a remote processing resource, particularly in the presence of privacy concerns and network latency issues.

There remains a need for systems and methods that balance the competing demands on storage, computation, and communications in the context of large machine learning models and continuous data feeds of high-resolution data.

### SUMMARY

A large foundation model is trained on a representative data set such as accumulated raw data from the sensors of a physiological monitor for a population of users. A distilled model is generated from the large foundation model and trained to identify information in a data stream from a physiological monitor that would be redundant or uninformative to the large foundation model. The distilled model can then execute on an edge device where it samples a data stream and filters out redundant or low-quality data from a source so that only portions of the data stream useful for inferencing are transmitted to the remote foundation model for processing.

In an aspect of the present disclosure, a system comprises: a remote processing resource hosting a foundation model trained on a large data set; a wearable monitor including one or more sensors, the wearable monitor configured to produce a data stream of continuous physiological data for a user based on signals from the one or more sensors; and a filter comprising a local model distilled from the foundation model, the local model trained to evaluate a data segment in the data stream from the wearable monitor and generate a control output based on whether the data segment contains high-quality data warranting transmission to the remote processing resource for analysis with the foundation model, and a switch responsive to the control output from the local model to selectively filter the data stream for communication to the remote processing resource based on data quality.

In some embodiments, high-quality data that warrants transmission may include new or useful information for the foundation model. In other embodiments, the switch may discard the data segment when the local model determines that the data segment contains redundant or low-quality information. According to some embodiments, the filter may execute on the wearable monitor itself. In other embodiments, the filter may execute on an edge device communicatively coupled between the wearable monitor and the remote processing resource. Moreover, in some implementations, the local model may sample the data stream at least once per second, where each data segment comprises one second of data. The data stream may include one or more of optical data, accelerometer data, gyroscope data, GeoPositioning System data, heart rate data, motion data, temperature data, or photoplethysmography data. The wearable monitor may include one or more of a wristband, a bicep band, a ring, a waistband, or a compression garment.

Implementations may include one or more of the following features. The switch may transmit the data segment of the data stream to the remote processing resource only after a predetermined duration of continuous new or high-quality data. The switch may store a representation of a prior segment of the data stream transmitted to the remote processing resource and evaluate the data segment to determine whether the data segment contains data different than the prior segment of the data stream. The remote processing resource may be configured to dynamically modify behavior of the switch based on the quality of data received from the wearable monitor. For example, the remote processing resource may provide a signal for the switch to apply a first threshold for transmitting data when the remote processing resource receives redundant or low-quality data, and a signal for the switch to apply a second threshold for transmitting data when the remote processing resource does not receive redundant or low-quality data. In some embodiments, the local model may use a small convolutional neural network, recurrent neural network, or transformer optimized for low power usage and trained to minimize cross-entropy between predictions by the local model and binary labels produced by the foundation model.

In an aspect, a computer program product disclosed herein comprises computer-executable code embodied in a non-transitory computer-readable medium that, when executing on one or more computing devices, causes the one or more computing devices to perform steps of: storing a local model in a memory of a wearable monitor, the local model distilled from a foundation model hosted on a remote processing resource and trained to evaluate whether a data segment of a data stream contains information useful for the foundation model to perform inferencing on the data stream; receiving the data stream as continuous physiological data from a user based on signals from one or more sensors of the wearable monitor; evaluating, with the local model, the data segment of the data stream to determine whether the data segment contains information useful for the foundation model to perform inferencing on the data stream, thereby providing an evaluation; and selectively filtering the data segment of the data stream for communication to the remote processing resource with a switch responsive to a transmit signal from the local model based on the evaluation. Other embodiments of this aspect may also or instead include a system having a wearable monitor and one or more processors configured to execute the foregoing steps. According to some embodiments, the method may further comprise receiving a modification of a threshold for the local model to provide the transmit signal from a feedback module of the remote processing resource. In some embodiments, the local model may be deployed on the wearable monitor or on a user device coupled between the wearable monitor and the remote processing resource.

In an aspect, a method for edge processing of physiological data comprises: storing a local model in a memory, the local model distilled from a foundation model hosted on a remote processing resource and trained to evaluate whether a data segment of a data stream contains information useful for the foundation model to perform inferencing on the data stream; receiving the data stream as continuous physiological data from a user based on signals from one or more sensors of a wearable monitor; evaluating, with the local model, the data segment of the data stream to determine whether the data segment contains information useful for the foundation model to perform inferencing on the data stream, thereby providing an evaluation; and selectively filtering the data segment of the data stream for communication to the remote processing resource with a switch responsive to a transmit signal from the local model based on the evaluation.

According to some embodiments, the monitor may include one or more of optical sensors, motion sensors, electrical sensors, thermal sensors, or location sensors. In other embodiments, the monitor may include a wearable fitness monitor, a heart rate monitor, or an Internet-of-Things device. In further implementations, the monitor may comprise an industrial monitor, an ambient condition monitor, an activity monitor, a weather monitor, a health monitor, an audio monitor, or a sleep monitor.

Implementations of the described techniques may include hardware, a method or process, computer software on a computer-accessible medium, and a system.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, with emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a system for edge processing data from a monitoring device.

FIG. 7 shows a method for edge processing data from a monitoring device.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally, with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as DRAM, SRAM, EDO RAM), and so forth. Although not depicted, any of the devices or components described herein may include a computer-readable medium or other memory for storing program instructions, data, and the like.

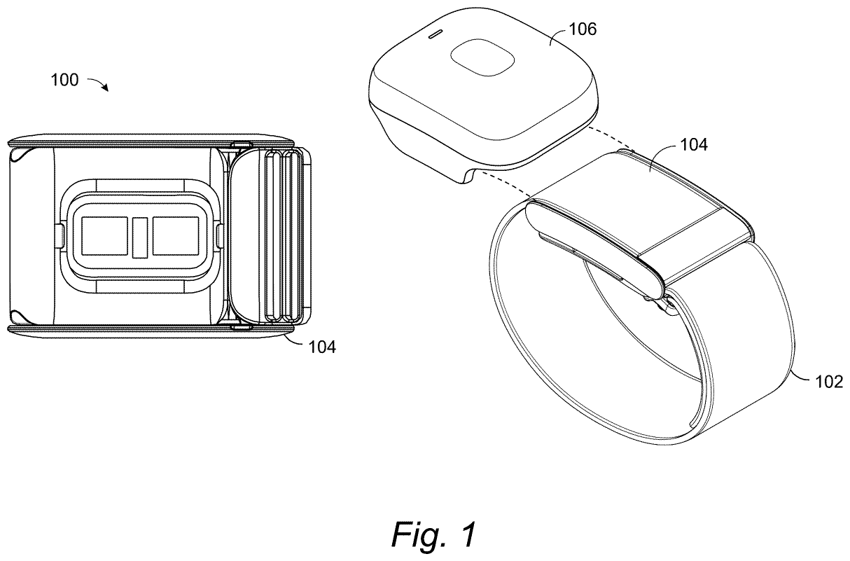

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104, such as data collection, processing, display, and communications with external resources. For example, the system 100 may include hardware for a heart rate monitor using, e.g., photoplethysmography, electrocardiogram any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light-emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters, including, but not limited to, skin temperature (using a thermometer), galvanic skin response (using a galvanic skin response sensor), motion (using one or more multi-axis accelerometers and/or gyroscope), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communications systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a Global Positioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

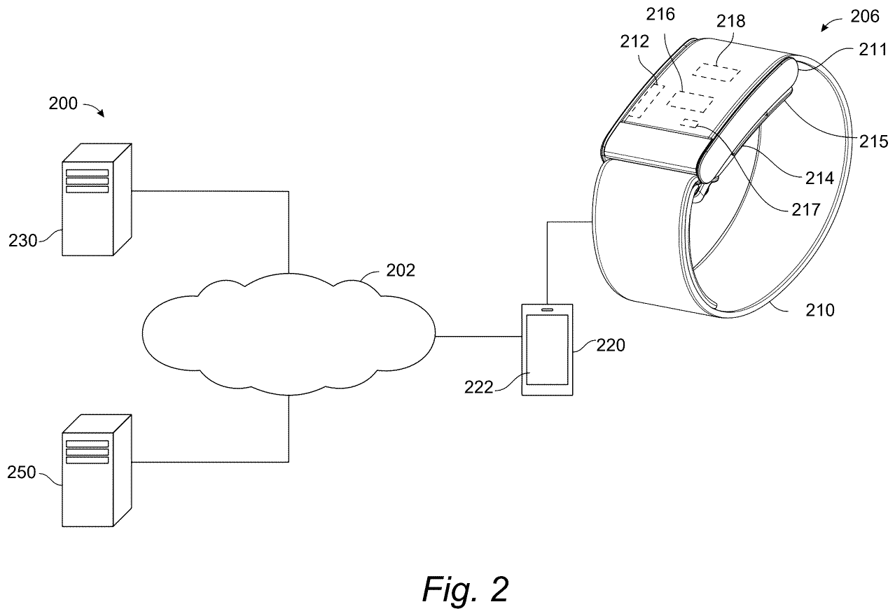

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communication with local resources. By way of non-limiting examples, short-range communications may include Wi-Fi communications, Bluetooth communications, infrared communications, near-field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

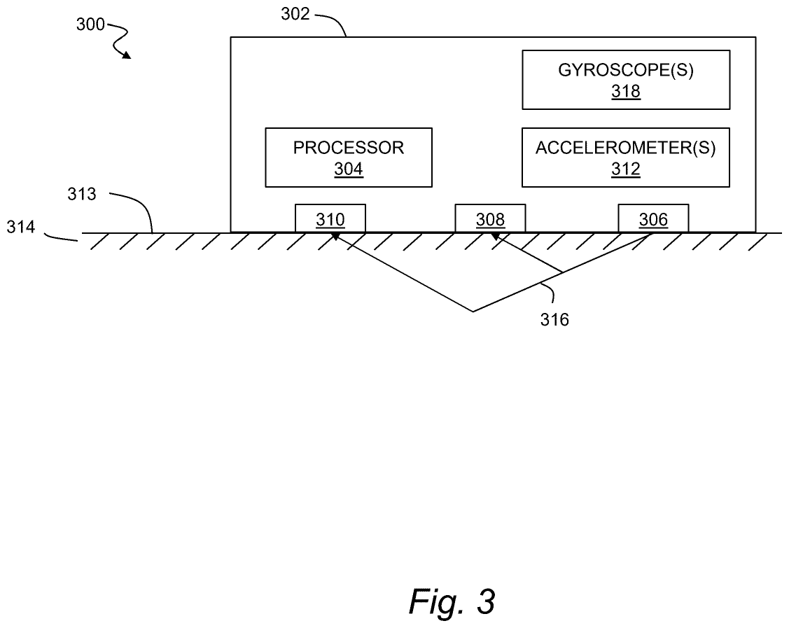

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light-emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light-emitting diodes that emit light in the green, red, infrared, near-infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308, 310 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, two sensors may be positioned at different distances from a perimeter of the physiological monitor 302 so that the sensors can acquire differential intensity values for ambient light incident on the skin and transmitted through the skin to the sensors.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

The physiological monitors described herein—e.g., in the systems 100, 200, 300 described above or elsewhere herein—may be provided in one or more different form factors.

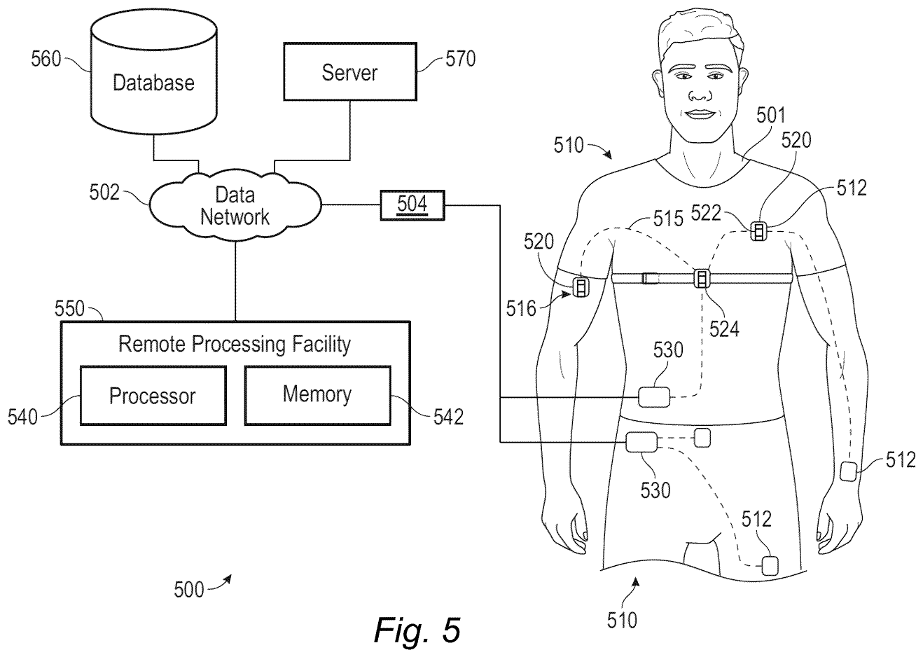

That is, although a wrist-worn device is illustrated in FIGS. 1 and 2, and garments with sensors are illustrated in FIG. 5, other form factors are also or instead possible, some of which are discussed below by way of example.

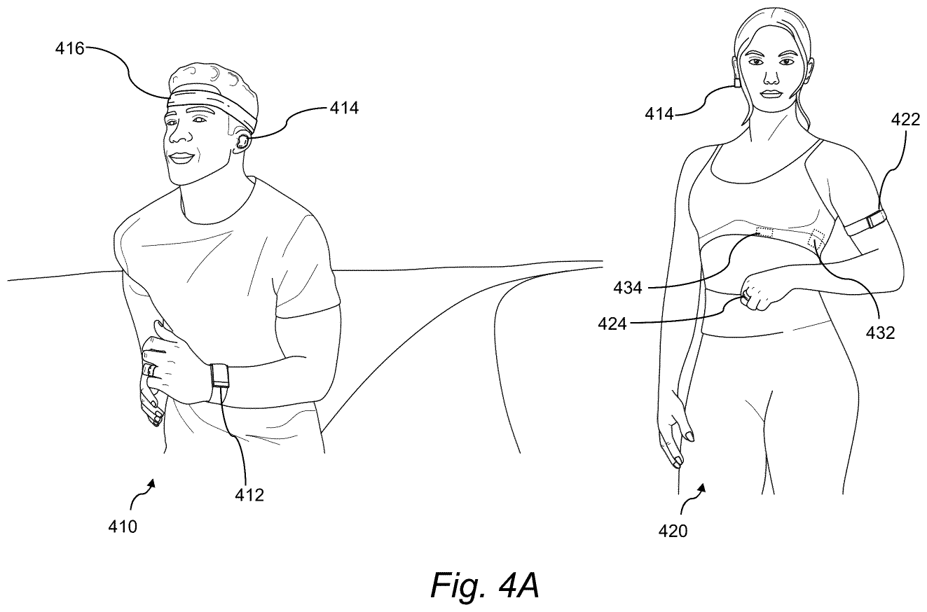

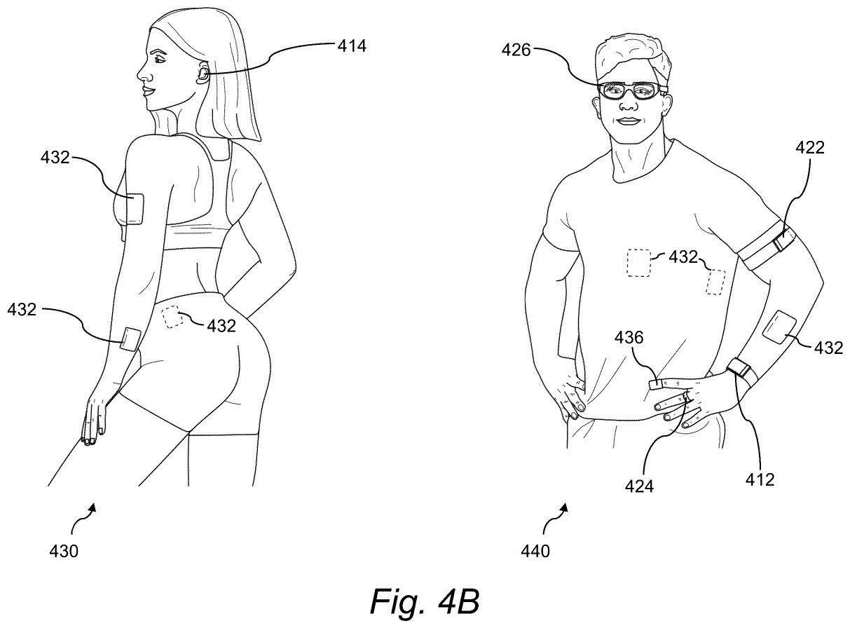

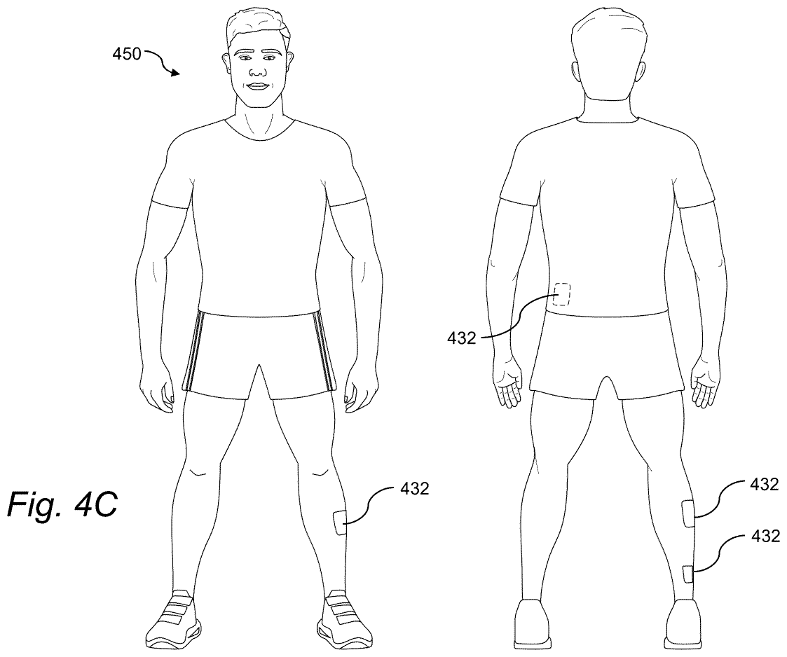

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring, such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health, and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes, that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wristbands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wristbands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiogram blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications, and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiogramalectroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for receiving light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements, such as accelerometer data from the module 520, which may be analyzed by a machine learning model or the like to infer a body position. In another aspect, this may be based on other signals from the module 520. For example, signals from sensors, such as photodiodes, temperature sensors, resistors, capacitors, and the like, may be used alone or in combination to infer a body position. In another aspect, the location may be determined based on the proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources, may advantageously be used in combination to improve physiological monitoring and achieve modes of monitoring not previously available.

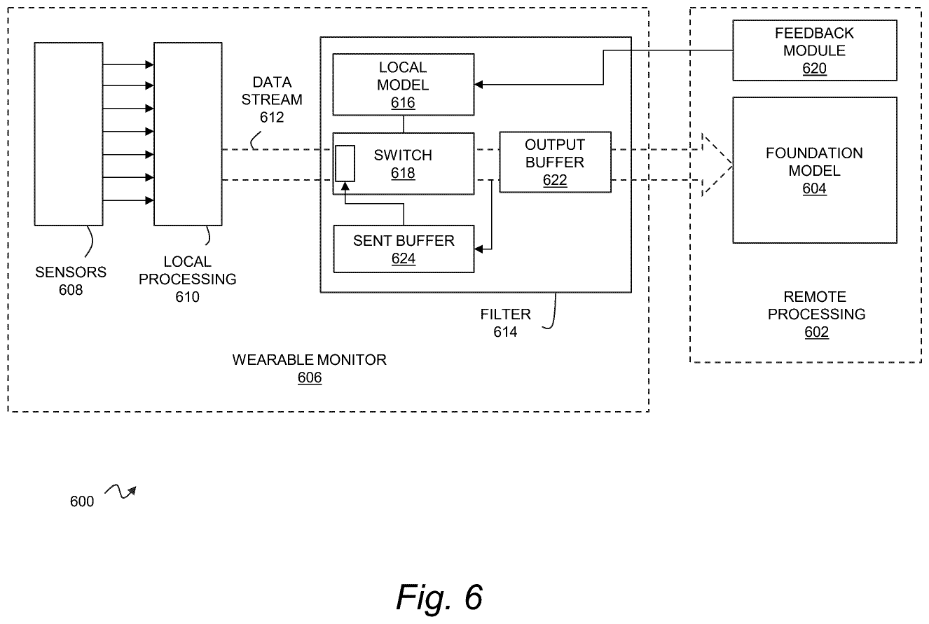

FIG. 6 shows a system for edge processing data from a monitoring device. In general, the system 600 may include a remote processing resource 602 hosting a foundation model 604 that has been trained on a large data set, a wearable monitor 606 or other monitoring device including one or more sensors 608 that has been configured with local processing 610 to produce a data stream 612 of continuous physiological data for a user based on signals from the one or more sensors 608; and a filter 614 including a local model 616 distilled from the foundation model 604, the local model 616 trained to evaluate a data segment in the data stream 612 from the wearable monitor 606 and generate a control output based on whether the data segment contains high-quality data that warrants transmission to the remote processing resource 602 for analysis with the foundation model 604, and a switch 618 responsive to the control output from the local model 616 to selectively filter the data stream 612 for communication to the remote processing resource 602 based on data quality. The remote processing resource 602 may also include a feedback module 620 that evaluates outputs or performance of the foundation model 604 and provides feedback signals to the local model 616 and/or switch 618 to modify filtering of the data stream 612 to the remote processing resource 602. The filter 614 may include other components such as an output buffer 622 to store data segments for batch transmission to the remote processing resource 602 and a sent buffer 624 that stores one or more most recent data segments in the data stream 612 to detect identical or substantially redundant sequential data in order to drop, compress, or otherwise preprocess data segments in the data stream 612 prior to transmission to the remote processing resource 602.

Although the emphasis in FIG. 6 is on a wearable monitor 606 that is worn by a subject and provides continuous physiological data, it will be understood that the techniques described herein may usefully be adapted to any context where one or more monitoring devices locally generate a stream of data, and a large foundation model has been trained to provide inferencing based on the corresponding data source(s). Thus, for example, the techniques described herein may usefully be employed with any monitor such as an Internet-of-Things device, a heart rate monitor, a smart home system, an automobile sensing network, an industrial monitor, a physiological monitor, an ambient condition monitor, an activity monitor, a weather monitor, a health monitor, an audio monitor, a sleep monitor, and so forth.

The remote processing resource 602 may include one or more servers, cloud computing resources, or other remote compute infrastructure configured to receive filtered data originating from the wearable monitor 606. In general, the remote processing resource 602 may provide compute and storage capabilities exceeding those available at the wearable monitor 606, and may be configured to execute computationally intensive analysis on received data, including inferencing using the foundation model 604 and/or representation learning, and to provide signals back to the wearable monitor 606 (e.g., via a feedback module 620) to influence edge-side filtering behavior. The remote processing resource 602 may also or instead include computing resources to train the foundation model 604, performing inferencing and related functions (e.g., agentic workflows, tool usage, data storage, and non-model processing, etc.) with the foundation model 604, and to support pre-training work for the foundation model 604, such as data acquisition, data cleansing, data labeling, and so forth. As further described herein, the remote processing resource 602 may also or instead support a web application interface for use of the foundation model 604 and related services and/or provide backend support for local user apps related to the wearable monitor 606, the foundation model 604, and so forth, including without limitation any of the server-based or remote processing tasks described herein.

The foundation model 604 may be a machine learning model hosted by the remote processing resource 602 and trained using a large corpus of data (e.g., unlabeled raw physiological signals and/or other sensor signals) to learn general-purpose representations and/or to support one or more downstream tasks. In some embodiments, the foundation model 604 is a self-supervised model that maps raw or lightly processed sensor signals into an embedding space suitable for tasks such as activity classification, anomaly detection, cardiac-related inference, or other analyses. The foundation model 604 may also generate supervisory signals used to train or refine the local model 616, such as novelty scores, quality scores, embeddings, binary labels, or other teacher outputs.

In some embodiments, the foundation model 604 may be trained using self-supervised learning on a large corpus of unlabeled physiological and contextual sensor data collected from wearable monitors across a population of users. For example, training may include masked-signal modeling, in which portions of a multi-modal time series (e.g., PPG, accelerometer, gyroscope, temperature, and/or ECG) are masked, and the model is trained to reconstruct the masked portions, thereby learning latent representations that capture physiological structure and cross-modal dependencies. Training may also or instead include feature extraction or other pre-processing of raw data in the data stream from monitors in order to facilitate improved detection of useful signals therein. As another example, training may include contrastive representation learning, in which temporally adjacent windows from the same session, user, or activity are treated as positive pairs, and other windows are treated as negative pairs, with an objective that increases similarity of embeddings for positive pairs while decreasing similarity for negative pairs. As another example, training may include an autoencoding objective (including variational autoencoding), in which an encoder maps sensor windows into a latent embedding, and a decoder reconstructs the input window (or a denoised version thereof), optionally with augmentation operations, such as time warping, random cropping, additive noise, amplitude scaling, channel dropout, and/or frequency-domain perturbations, to improve robustness to motion artifacts, sensor displacement, ambient light interference, and device-to-device variability.

In some embodiments, the foundation model 604 may additionally or alternatively be trained using supervised learning, weak supervision, and/or multi-task learning to improve performance on downstream physiological inferences while preserving general-purpose representations learned from large-scale data. For example, training may use labeled outcomes (e.g., activity classes, sleep stages, arrhythmia events, recovery metrics, or clinically-adjudicated labels) with cross-entropy, focal loss, or regression losses, optionally with class rebalancing and hard-example mining to address label sparsity and skew. As another example, training may use pseudo-labeling in which a preliminary model (or an ensemble) generates pseudo-labels for unlabeled sensor segments and the foundation model is iteratively refined using the pseudo-labels, optionally with confidence thresholding. As another example, training may employ knowledge distillation in which teacher outputs (e.g., embeddings, quality scores, novelty scores, uncertainty estimates, or logits for one or more tasks) provide supervision to a student model during pretraining or fine-tuning, and may employ domain-adaptation techniques such as device-identifier conditioning, adversarial invariance objectives, normalization calibrated by device type, and/or per-user personalization layers to improve generalization across different wearable form factors, sensor modalities, sampling rates, and user populations.

The wearable monitor 606 may be any wearable physiological monitoring device configured for continuous or substantially continuous acquisition of user data, such as a wrist-worn device, bicep band, ring, patch, garment-integrated module, or other monitor. The wearable monitor 606 may include sensing, processing, memory, and communications capabilities sufficient to generate a data stream 612 and to execute edge-side functions including conversion of raw sensor data with local processing 610 and filtering with the filter 614. In general, the local processing may include any suitable filtering, digitization, data cleaning, gain control, and so forth suitable for converting analog front-end signals into a digital data stream suitable for use in subsequent processing. As described herein, the wearable monitor 606 may be configured to transmit selected portions of the data stream 612 to the remote processing resource 602 based on control outputs generated locally (e.g., by the local model 616) and/or based on feedback from the remote processing resource 602.

The sensors 608 may include one or more physiological and/or contextual sensors coupled to the wearable monitor 606 and configured to generate raw sensor signals. By way of example and not limitation, the sensors 608 may include optical sensing components for photoplethysmography, inertial sensors such as accelerometers and gyroscopes, electrical sensors such as electrodes for ECG or related measurements, thermal sensors for temperature, location sensors such as GeoPositioning System (GPS) sensors, and so forth. The sensors 608 may also or instead include one or more optical sensors, one or more motion sensors, one or more electrical sensors, one or more thermal sensors, one or more location sensors, and so forth. Outputs from the sensors 608 may be digitally sampled at any suitable rate by local processing 610 to form all or part of the data stream 612.

The local processing 610 may include one or more signal-processing operations performed at or near the wearable monitor 606 prior to filtering and/or transmission. For example, local processing 610 may include sampling, time-windowing into data segments, synchronization across sensor modalities, denoising, motion artifact reduction, normalization, calibration, feature extraction, compression, formatting, buffering, and/or packaging of sensor data. In some embodiments, local processing 610 prepares one-second windows (or other durations) of multi-modal data for evaluation by the local model 616 and for potential storage in buffers (e.g., output buffer 622 and/or sent buffer 624).

The data stream 612 may include a continuous or substantially continuous sequence of sensor-derived data produced by the wearable monitor 606 and processed into a stream of data by local processing 610. The data stream 612 may be segmented into data segments (e.g., fixed-duration windows) that are evaluated for novelty, quality, redundancy, or other criteria associated with usefulness to the foundation model 604 as described herein. The data stream 612 may include one or more modalities, such as PPG waveforms, motion signals, temperature signals, and/or other physiological or contextual signals, and may be selectively transmitted in whole or in part to the remote processing resource 602 based on decisions performed by the filter 614. Thus, the data stream may include descriptive data such as photoplethysmography data, heart rate data, motion data, temperature data, or more generally, raw physical data from any suitable sensors such as optical data, accelerometer data, gyroscope data, electrical data, and so forth.

The filter 614 may include hardware, software, or a combination thereof configured to control whether and how portions of the data stream 612 are transmitted from the wearable monitor 606 to the remote processing resource 602. In general, the filter 614 may operate in real time or near-real time to evaluate successive data segments and selectively forward data that is predicted to be useful for inferencing by the foundation model 604 while suppressing, compressing, summarizing, or discarding other data, generally based on control signals obtained from the local model 616. As depicted, the filter 614 may also include the switch 618, the output buffer 622, and the sent buffer 624, which may cooperate to control filtering of the data stream 612. The filter 614 may also cooperate with the feedback module 620 to adapt filtering behavior over time based on an analysis of the data stream 612 received by the remote processing resource 602. Although depicted as executing on a wearable monitor 606, it will be appreciated that the filter 614 may also or instead be deployed and/or execute on any edge device(s) communicatively coupled between the wearable monitor 606 and the remote processing resource 602. Thus, for example, the filter 614 may be deployed on a smartphone or other user device described herein that connects the wearable monitor 606 to the remote processing resource 602 so that this intermediate device can apply the filtering techniques described herein before data is sent over a data network to the remote processing resource 602.

The filter 614 may be used with a wearable monitor; however, it will be understood that the filter architecture described herein may more generally be applicable to any system using a local monitor and a remote foundation model trained on data typical of the local monitor. Thus, in one aspect, a filter 614 described herein includes an input to the filter, the input configured to receive a data stream of continuous physiological data; a local model stored in a memory of the filter, the local model distilled from a foundation model and the local model trained to: determine whether a portion of the data stream contains information useful for inferencing on the data stream of continuous physiological data with the foundation model, and generate a control signal for controlling transmission of the portion of the data stream to the foundation model based on an evaluation from the local model; a switch coupled to an output of the filter; and a processor configured by computer-executable code to receive the control signal from the local model and to responsively control the switch to transmit the portion of the data stream from the output of the filter to a remote processing resource hosting the foundation model. In another aspect, a system using such a filter may include a monitor including one or more sensors, the monitor configured to produce a data stream of continuous data from a monitored item based on signals from the one or more sensors; and a filter comprising: a local model distilled from a foundation model, the local model trained to evaluate a segment in the data stream from the monitor and generate a control output based on whether the segment contains high-quality data useful for the foundation model to perform inferencing on the data stream, and a switch responsive to the control output from the local model to selectively filter the segment of the data stream for communication to a remote processing resource hosting the foundation model based on whether the segment contains high-quality data useful for the foundation model to perform inferencing on the data stream.

The local model 616 (also referred to herein as a “distilled model” or “distilled local model”) may be a distilled and/or otherwise compressed model derived from the foundation model 604 and configured to execute within edge constraints of the wearable monitor 606. The local model 616 may sample the data stream 612 at any suitable rate, e.g., at least once per second and including at least one second of data from the wearable monitor 606, or at any other interval suitable for the processing capabilities of the wearable monitor 606, the processing capabilities of the remote processing resource 602, and so forth. For example, the local model 616 may receive, as input, a data segment of the data stream 612 (e.g., a one-second window of multi-modal sensor data) and generate a control output indicative of whether the data segment should be transmitted, deferred for batching, summarized, compressed, or discarded. For example, the local model may be trained to evaluate a data segment in the data stream from the wearable monitor and generate a control output based on whether the data segment contains high-quality data that warrants transmission to the remote processing resource for analysis with the foundation model. In this context, high-quality data may, for example, include new or useful information for the foundation model 604. In some embodiments, the local model 616 outputs a binary control signal, while in other embodiments the local model 616 outputs a score (e.g., novelty and/or quality) that is compared to one or more thresholds to form a transmission decision, optionally incorporating comparisons to prior sent content (e.g., via the sent buffer 624) and/or remote feedback. The local model 616 may use a variety of techniques to compress and encode the foundation model 604. For example, the local model 616 may use a small convolutional neural network, recurrent neural network, or transformer optimized for low power usage and trained to minimize cross-entropy between predictions by the local model and binary labels produced by the foundation model.

In some embodiments, the local model 616 is trained as a student model using knowledge distillation from the foundation model as a teacher. A training corpus may be formed from historical multi-modal sensor recordings collected from a population of wearable monitors, and may be segmented into fixed-duration windows (e.g., 0.5 second, 1 second, 2 second, 5 second, or 10 second windows) with optional overlap. For each window, the foundation model may be executed to generate one or more supervisory signals that characterize the usefulness of the window for backend inferencing, such as a novelty score relative to prior context, a signal quality score, an uncertainty estimate, an embedding distance to one or more reference embeddings, a task-specific confidence (e.g., for activity classification, arrhythmia detection, or anomaly detection), or a composite score combining two or more of the foregoing. In some embodiments, teacher outputs are converted into student targets by binarization (e.g., transmit versus discard), where a label is assigned based on whether the teacher output satisfies one or more criteria, including exceeding a novelty threshold, exceeding a quality threshold, differing from a prior transmitted embedding by more than a distance threshold, and/or satisfying a rule that requires a plurality of consecutive windows to meet a criterion before labeling a batch for transmission. In some embodiments, labels are refined using additional metadata, including device type, sampling rate, motion state, skin contact state, ambient light level, and/or user activity state, thereby producing context-conditioned targets that teach the local model to be more selective in conditions associated with predictable artifacts.

In some embodiments, the local model 616 is trained to operate under edge constraints and to output a control value suitable for directly driving a transmission policy. For example, the local model 616 may be trained to output a probability of transmission, a novelty score, a quality score, or a multi-dimensional control vector that includes a transmit decision and an action selection (e.g., transmit raw, transmit compressed, transmit features only, buffer for batch, or discard). Training may minimize a distillation loss between student outputs and teacher targets, such as cross-entropy for a binary transmit label, Kullback-Leibler divergence for matching soft targets, and/or regression losses for matching continuous teacher scores, optionally combined with regularization terms that encourage low compute and stable decisions across time. In some embodiments, the training objective includes a communications-aware penalty that biases the student toward fewer transmissions, such as a term proportional to an expected transmission rate and/or an estimated transmission energy, thereby directly optimizing a tradeoff between agreement with the teacher and edge power consumption. In some embodiments, the student is trained with augmentations that simulate wearable conditions (e.g., motion artifacts, sensor displacement, dropped samples, quantization noise, or changes in skin tone and perfusion) so that the student learns robust transmit decisions in noisy settings, and the trained student is then optimized for deployment via quantization (e.g., 8-bit integer quantization), pruning, architecture search, and/or compilation for a target microcontroller or edge accelerator.

The switch 618 may include a hardware and/or software-controlled gate responsive to the control output from the local model 616 to selectively pass or block corresponding portions of the data stream 612. In some embodiments, the switch 618 forwards a data segment when the local model 616 indicates that the data segment contains sufficiently novel and/or high-quality information, and otherwise blocks transmission of the data segment or routes the data segment to local handling (e.g., local discard, local summarization, or local compression). Conversely, the switch 618 may discard a data segment when the local model 616 determines that the data segment contains redundant or low-quality information. The switch 618 may also support conditional forwarding, such as transmitting only after a predetermined number of consecutive segments meet a criterion, or transmitting as a batch from the output buffer 622.

A variety of techniques may be used to evaluate the novelty or quality of data segment information in this context. In some embodiments, novelty of information in a data segment is evaluated as a measure of dissimilarity between the segment and prior context, including one or more previously transmitted segments stored in a sent buffer. In one approach, the local model and/or foundation model maps a segment into an embedding space, and novelty is computed from an embedding distance between a current embedding and one or more reference embeddings representing recently sent segments, a rolling baseline, or an activity-conditioned centroid, where larger distances indicate greater novelty. In another approach, novelty is computed from a predictive or reconstruction error, such as a residual produced when a predictive model forecasts the segment from prior segments, or an error produced when an autoencoder reconstructs the segment, where larger errors indicate a deviation from expected patterns. In another approach, novelty is computed using a likelihood or density score (e.g., a negative log-likelihood under a learned generative model), an entropy score, or an uncertainty score, where low likelihood or high uncertainty indicates that the segment is atypical relative to previously observed data. In some embodiments, novelty is further conditioned on state information such as detected activity, device placement, and/or time of day, so that novelty is assessed relative to an appropriate baseline for the current state, and the novelty decision is stabilized using hysteresis, change-point detection, and/or a rule requiring a plurality of consecutive segments to exceed a novelty threshold before initiating transmission or forming a batch for transmission.

In some embodiments, novelty and/or quality of a data segment is evaluated in terms of an expected utility of the data segment for inferencing by the foundation model, where “useful” information includes information that is expected to change, refine, or disambiguate an inference produced by the foundation model as compared to inferencing performed without the data segment. In one aspect, usefulness may be represented by a teacher signal generated by executing the foundation model on the data segment (optionally in view of prior context), and measuring a marginal contribution of the data segment to one or more foundation-model outputs. For example, a usefulness score may be based on a change in an embedding produced by the foundation model when the data segment is appended to a prior sequence, a change in a task-specific output (e.g., an activity class distribution, an anomaly score, an arrhythmia likelihood, a recovery metric, or a sleep-stage probability), a reduction in predictive uncertainty (e.g., a reduction in entropy of a posterior distribution or a reduction in variance across an ensemble), or a change in a calibrated confidence metric. In this manner, “novelty” may correspond to a data segment that moves a foundation-model representation and/or output beyond a threshold relative to a baseline formed from previously processed or previously transmitted segments, and “quality” may correspond to a data segment that yields stable, internally consistent, and low-noise representations for which the foundation model exhibits higher confidence and/or lower reconstruction residuals.

In some embodiments, usefulness is quantified using a proxy for information gain with respect to the foundation model's internal representations. For example, the foundation model may be configured to produce (or permit computation of) a reconstruction loss for a masked-signal objective, a predictive loss for forecasting a subsequent segment, and/or an out-of-distribution score for the data segment, and the data segment may be considered more useful when the loss and/or out-of-distribution score indicates that the segment is not well explained by previously observed data. In another aspect, usefulness may be based on a distance in embedding space between an embedding for the data segment and one or more embeddings for previously transmitted segments, where the distance is optionally normalized or conditioned based on an inferred state (e.g., detected activity, device placement, motion state, or time of day) so that a segment is deemed useful when it is atypical for a current state. In another aspect, usefulness may be evaluated based on an expected change in one or more downstream probe tasks used to validate general-purpose representations of the foundation model, such as whether including the data segment causes a materially different predicted activity label, gait abnormality score, cardiac-event score, or similar output, thereby indicating that the segment carries discriminative information for the foundation model.

In some embodiments, quality is evaluated as usefulness conditioned on a reliability of the data segment for the foundation model, e.g., based on characteristics of the locally measured sensor signals, such that a data segment is downgraded when artifacts are expected to cause the foundation model to produce embeddings or outputs that are unstable, misleading, or non-informative. For example, the data segment may be deemed low quality for inferencing when motion metrics, ambient-light leakage metrics, clipping metrics, missing-sample metrics, or electrode impedance metrics indicate sensor corruption, and the expected utility is thereby reduced because the foundation model would be likely to ignore the segment, treat it as noise, or produce a high-uncertainty output. In some embodiments, a composite utility score is computed that combines (i) a novelty component representing an expected change in foundation-model representation or output and (ii) a quality component representing an expected reliability of that change, and the composite utility score is compared to one or more thresholds to select among actions including transmitting raw samples, transmitting a compressed representation, buffering for batch transmission, or discarding the data segment.

In some embodiments, usefulness is evaluated online by the remote processing resource 602 after receipt of transmitted segments, and feedback is generated to better align edge-side decisions with inferencing utility of the foundation model 604. For example, the remote processing resource 602 may tag a received segment as low utility when the foundation model 604 produces an embedding substantially similar to embeddings for recently received data, when the segment fails to change a task output beyond a change threshold, when the segment increases uncertainty, or when the segment is identified as corrupted by a model-based quality detector. Conversely, the remote processing resource 602 may tag a received segment as high utility when it materially changes a predicted state, reduces uncertainty, triggers detection of an event, or improves consistency of a temporal inference over a surrounding interval, and the remote processing resource 602 may responsively adjust one or more thresholds, decision parameters, or distilled-model updates used at the edge to increase transmission of similarly useful segments and suppress transmission of similarly unhelpful segments.

The feedback module 620 may include logic executing at the remote processing resource 602 to evaluate the usefulness of data received from the wearable monitor 606 and to provide feedback that modifies edge-side filtering behavior. Through the feedback module 620, the remote processing resource 602 may be configured to dynamically modify behavior of the switch 618 based on a quality of data received from the wearable monitor 606. For example, the feedback module 620 may analyze outputs of the foundation model 604 to determine whether received segments were redundant, low quality, or otherwise not useful, and responsively transmit an instruction to adjust one or more parameters at the wearable monitor 606, such as a novelty threshold, a quality threshold, a batching policy, a false-positive/false-negative tradeoff parameter, or other decision criteria. Thus, in one aspect, the remote processing resource 602 may signal the switch 618 (through the feedback module 620) to apply a first threshold for transmitting data when the remote processing resource 602 receives redundant or low-quality data, and the remote processing resource 602 may signal the switch 618 to apply a second threshold for transmitting data when the remote processing resource 602 does not receive redundant or low-quality data. In some embodiments, the feedback module 620 may also or instead support periodic retraining and/or updating of the local model 616 based on newly observed data distributions or performance characteristics.

The output buffer 622 may include memory configured to accumulate data segments that have been selected for transmission so that they may be sent in a batch, in a burst, or according to an availability of network resources or power constraints. For example, the output buffer 622 may store a rolling set of consecutive segments that meet a novelty and/or quality threshold until a batch size or time interval criterion is satisfied, after which the stored batch is transmitted to the remote processing resource 602. This permits the switch 618 to forward data segments in the data stream 612 only after a predetermined duration of continuous new or high-quality data. Buffering may also more generally reduce communications overhead, permit opportunistic transmission, and provide a mechanism to enforce policies such as “send only after a predetermined duration of continuous high-quality data” or “send at least once per minute.”

The sent buffer 624 may include memory configured to store information describing one or more previously transmitted data segments and to support redundancy detection and/or novelty assessment for subsequent segments. In some embodiments, the sent buffer 624 stores raw data, compressed representations, features, and/or embeddings corresponding to recently transmitted segments, and the wearable monitor 606 uses these stored representations to determine whether newly observed data is identical or substantially similar to previously sent content. The sent buffer 624 may thus support dropping or suppressing repetitive segments, transmitting deltas relative to prior segments, and/or supplying comparison inputs to the local model 616 and/or other decision logic within the filter 614.

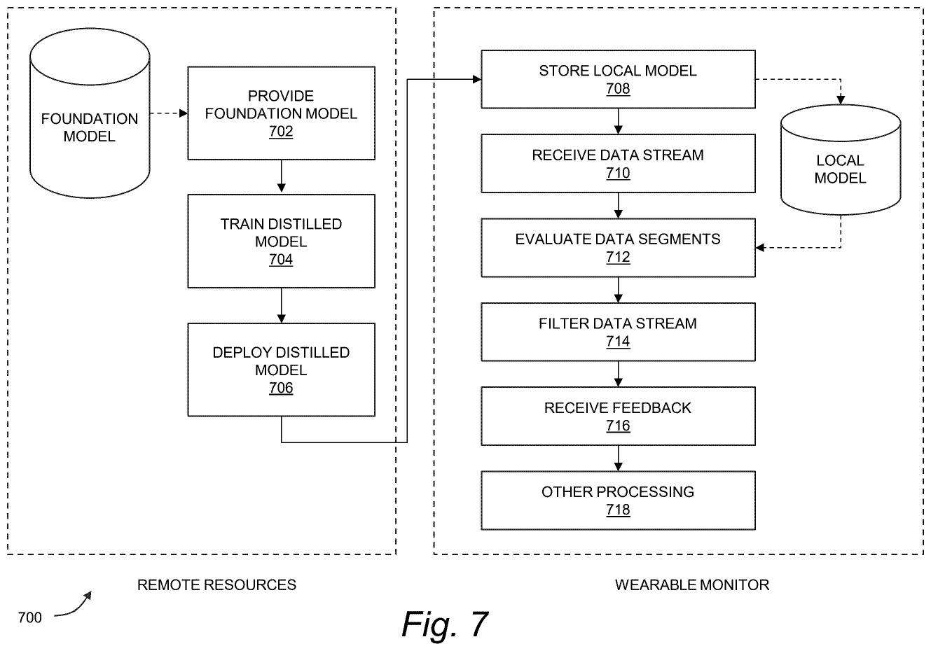

FIG. 7 shows a method for edge processing data from a monitoring device. In one aspect, some steps (e.g., steps 702-706) of the method 700 may be performed by remote resources, such as any of the servers or other computing resources described herein, while other steps (e.g., steps 708-716) are performed by a wearable physiological monitor or similar device described herein. As a significant advantage, the method 700 may be used with a wearable device to reduce bandwidth, power consumption, and latency by selectively filtering out data segments of a continuous data stream for communication to a remote resource while preserving data segments that contain information useful for inferencing by a foundation model.

As shown in step 702, the method 700 may include providing a foundation model. In one aspect, this may include training the foundation model with the remote resources. In another aspect, the foundation model may be obtained from a third party for use in monitoring as described herein. The foundation model may include any of the foundation models described herein. For example, the foundation model may be trained on a large data set of physiological and/or contextual sensor data, and may be configured to perform inferencing on a data stream from a wearable monitor, including generation of embeddings, prediction of physiological metrics, activity classification, anomaly detection, or other downstream analyses. In some embodiments, providing the foundation model includes training and/or maintaining the foundation model, storing model parameters in memory accessible to a remote processing resource, and configuring one or more network-accessible services that permit inferencing with the foundation model on data received from one or more wearable monitors.

As shown in step 704, the method 700 may include training a distilled model (also referred to herein as a “local model”) based on the foundation model. In some embodiments, training includes knowledge distillation, in which the foundation model provides supervisory signals, labels, scores, embeddings, or other teacher outputs for training the distilled model to evaluate whether a data segment of a data stream contains information useful for the foundation model to perform inferencing on the data stream. The distilled model may be trained to generate a transmit signal and/or other control output indicative of whether a corresponding data segment should be communicated to the remote resources, and the training may include optimizing a tradeoff between agreement with the foundation model and one or more edge constraints, such as compute, memory, bandwidth, and power consumption.

As shown in step 706, the method 700 may include deploying the distilled model from the remote resources to a wearable monitor. In some embodiments, deploying includes transmitting model parameters and/or executable instructions for the distilled model to the wearable monitor and provisioning one or more configuration values for operation of the distilled model on the wearable monitor.

As shown in step 708, the method 700 may include storing a local model, e.g., the distilled model derived from the foundation model, in a memory of the wearable monitor. As described herein, the distilled model or local model may be trained to evaluate whether a data segment of a data stream contains information useful for the foundation model to perform inferencing on the data stream.

As shown in step 710, the method 700 may include receiving, at the wearable monitor, a data stream as continuous data based on signals from one or more sensors of the wearable monitor. In some embodiments, the data stream includes continuous physiological data from a user, and the wearable monitor may segment the data stream into data segments for subsequent evaluation by the local model.

As shown in step 712, the method 700 may include evaluating, with the local model on the wearable monitor, a data segment of the data stream to determine whether the data segment contains information useful for the foundation model to perform inferencing on the data stream. In some embodiments, evaluating a data segment includes generating, with the local model, a transmit signal for a switch based on the determination of whether the data segment contains useful information for the foundation model to perform inferencing on the data stream.

As shown in step 714, the method 700 may include selectively filtering the data segment of the data stream for communication to the remote processing resource with a switch responsive to the transmit signal from the local model based on the determination from the local model of whether the data segment contains useful information for the foundation model to perform inferencing on the data stream. In some embodiments, selectively filtering includes transmitting the data segment when the transmit signal indicates transmission (e.g., a high-quality or high-information data segment) and suppressing transmission of the data segment when the transmit signal indicates non-transmission.

As shown in step 716, the method 700 may include receiving, at the wearable monitor, feedback from the remote resources. In some embodiments, the feedback includes a modification of a threshold for the local model to provide the transmit signal to the switch, where the modification is based on an assessment by the remote resources of the usefulness of previously transmitted data segments for inferencing by the foundation model. In this manner, the remote resource may monitor actual inferencing results based on the current data stream, and provide feedback to the filter on the wearable monitor to increase data transmission (e.g., when most or all of the transmitted data is useful for inferencing) or decrease data transmission (e.g., when little or no transmitted data is useful for inferencing).

As shown in step 718, the method 700 may include performing other processing. For example, other processing may include local processing at the wearable monitor such as buffering, compression, summarization, storage, user-interface updates, or power management, and/or remote processing such as inferencing with the foundation model on received data segments, storage of received data segments, retraining of one or more models, and generation of additional feedback to the wearable monitor. It will also be appreciated that, while the other processing is illustrated as occurring on the wearable monitor, useful additional processing may also or instead be performed with the remote resources or other computing resources associated with the disclosed system. Thus, more generally, a wide range of other processing may also or instead be performed with or based on the data stream and resulting outputs from the foundation model.

By way of non-limiting examples, remote resources may execute the foundation model on received data segments to generate embeddings, novelty scores, quality scores, anomaly scores, activity classifications, sleep-stage classifications, cardiac event likelihoods, recovery metrics, strain metrics, respiration estimates, blood oxygen estimates, blood pressure estimates, or other physiological inferences, and may store the received data segments and/or inferred outputs in a database for subsequent longitudinal analysis, model improvement, personalization, and user reporting. The remote resources may also aggregate inferred outputs over time to detect trends, perform change-point detection, compute baselines, identify outliers, generate alerts, and populate user-interface content for presentation at a user device, including recommendations, coaching, or notifications triggered by the inferred outputs of the foundation model. As a significant technical advantage, these and other inferencing functions can be improved by using the filter described herein to reduce computational overhead by reducing the volume of data in the data stream, and improving the speed and accuracy of outputs by improving the quality (e.g., the inferencing value) of the data stream.

As further examples, the remote resources may use the inferred outputs to adapt operation of the wearable monitor by generating feedback messages that update one or more thresholds, policies, or configuration parameters for the local model and/or the switch, including thresholds for novelty, quality, and/or utility, a minimum number of consecutive qualifying segments to buffer before transmission, a maximum transmit rate, a compression mode, or a selection among different local models or parameter sets based on device state. In some embodiments, the feedback is based on a measured marginal utility of recently transmitted data segments for inferencing by the foundation model, such as whether the transmitted segments materially changed an embedding trajectory, altered a task output beyond a change threshold, reduced uncertainty, triggered event detection, or were determined to be redundant or corrupted, and the remote resources may responsively adjust the feedback to reduce redundant transmissions or to reduce missed high-utility segments. In some embodiments, the remote resources may also update or retrain the local model based on the received data stream, and deploy an updated local model or updated parameters to the wearable monitor.

As further examples, the wearable monitor may perform local post-processing using the data segments and/or received feedback. For example, the wearable monitor may buffer selected segments for opportunistic transmission when connectivity improves, batch segments according to a transmit schedule, apply compression (including delta coding relative to a stored prior segment) based on a compression policy received in feedback, or locally summarize discarded segments into features or statistics for later transmission or local use. In some embodiments, the wearable monitor uses feedback to modify a local transmit decision policy, including adjusting thresholds, enabling hysteresis, changing segment durations, changing sampling rates, changing a modality-selection policy (e.g., transmitting only certain channels or transmitting additional modalities when a triggering condition occurs), and/or changing an on-device power mode. The wearable monitor may also update user-facing outputs locally, such as displaying current activity state, heart rate metrics, quality indicators, connectivity status, or transmission status, and may generate local alerts based on either locally computed metrics or on results returned from the remote resources following foundation-model inferencing.

More generally, any processing, analysis, display, coaching, feedback, tuning, or other functions may usefully be included as other processing for a monitor or remote resources in a method 700 as described herein.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software.

Thus, in one aspect, each method described above, and combinations thereof, may be embodied in computer-executable code that, when executing on one or more computing devices, performs the steps thereof. In another aspect, the methods may be embodied in systems that perform the steps thereof, and may be distributed across devices in a number of ways, or all of the functionalities may be integrated into a dedicated, standalone device or other hardware. The code may be stored in a non-transitory fashion in a computer memory, which may be a memory from which the program executes (such as random access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine, to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

1. A system comprising:
a remote processing resource hosting a foundation model trained on a large data set;
a wearable monitor including one or more sensors, the wearable monitor configured to produce a data stream of continuous physiological data for a user based on signals from the one or more sensors; and
a filter comprising:
a local model distilled from the foundation model, the local model trained to evaluate a data segment in the data stream from the wearable monitor and generate a control output based on whether the data segment contains high-quality data that warrants transmission to the remote processing resource for analysis with the foundation model, and
a switch responsive to the control output from the local model to selectively filter the data stream for communication to the remote processing resource based on data quality.

2. The system of claim 1, wherein the high-quality data that warrants the transmission includes new or useful information for the foundation model.

3. The system of claim 1, wherein the switch discards the data segment when the local model determines that the data segment contains redundant or low-quality information.

4. The system of claim 1, wherein the filter executes on the wearable monitor.

5. The system of claim 1, wherein the filter executes on an edge device communicatively coupled between the wearable monitor and the remote processing resource.

6. The system of claim 1, wherein the local model samples the data stream at least once per second.

7. The system of claim 1, wherein the data segment includes one second of data from the wearable monitor.

8. The system of claim 1, wherein the data stream includes one or more of optical data, accelerometer data, gyroscope data, and GeoPositioning System data.

9. The system of claim 1, wherein the data stream includes one or more of heart rate data, motion data, and temperature data.

10. The system of claim 1, wherein the data stream includes photoplethysmography data.

11. The system of claim 1, wherein the wearable monitor includes one or more of a wristband, a bicep band, a ring, a waistband, and a compression garment.

12. The system of claim 1, wherein the switch transmits the data segment of the data stream to the remote processing resource only after a predetermined duration of continuous new or high-quality data.

13. The system of claim 1, wherein the switch stores a representation of a prior segment of the data stream transmitted to the remote processing resource and evaluates the data segment to determine whether the data segment contains data different than the prior segment of the data stream.

14. The system of claim 1, wherein the remote processing resource is configured to dynamically modify behavior of the switch based on a quality of data received from the wearable monitor.

15. The system of claim 14, wherein the remote processing resource provides a first signal for the switch to apply a first threshold for transmitting the data when the remote processing resource receives redundant or low-quality data; and wherein the remote processing resource provides a second signal for the switch to apply a second threshold for transmitting the data when the remote processing resource does not receive the redundant or low-quality data.

16. The system of claim 1, wherein the local model uses a small convolutional neural network, recurrent neural network, or transformer optimized for low power usage and trained to minimize cross-entropy between predictions by the local model and binary labels produced by the foundation model.

17. A computer program product comprising computer-executable code embodied in a non-transitory computer-readable medium that, when executing on one or more computing devices, causes the one or more computing devices to perform steps of:
storing a local model in a memory of a wearable monitor, the local model distilled from a foundation model hosted on a remote processing resource and trained to evaluate whether a data segment of a data stream contains information useful for the foundation model to perform inferencing on the data stream;
receiving the data stream as continuous physiological data from a user based on signals from one or more sensors of the wearable monitor;
evaluating, with the local model, the data segment of the data stream to determine whether the data segment contains the information useful for the foundation model to perform the inferencing on the data stream, thereby providing an evaluation; and
selectively filtering the data segment of the data stream for communication to the remote processing resource with a switch responsive to a transmit signal from the local model based on the evaluation from the local model of whether the data segment contains the information useful for the foundation model to perform the inferencing on the data stream.

18. A method for edge processing of physiological data, the method comprising:
storing a local model in a memory, the local model distilled from a foundation model hosted on a remote processing resource and trained to evaluate whether a data segment of a data stream contains information useful for the foundation model to perform inferencing on the data stream;
receiving the data stream as continuous physiological data from a user based on signals from one or more sensors of a wearable monitor;
evaluating, with the local model, the data segment of the data stream to determine whether the data segment contains the information useful for the foundation model to perform the inferencing on the data stream, thereby providing an evaluation; and
selectively filtering the data segment of the data stream for communication to the remote processing resource with a switch responsive to a transmit signal from the local model based on the evaluation from the local model of whether the data segment contains the information useful for the foundation model to perform the inferencing on the data stream.

19. The method of claim 18, further comprising receiving a modification of a threshold for the local model to provide the transmit signal from a feedback module of the remote processing resource.

20. The method of claim 19, wherein the local model is deployed on the wearable monitor or a user device coupled between the wearable monitor and the remote processing resource.

21-35. (canceled)
